(19) United States
(12) Patent Application Publication
(10) Pub. No.: US 2008/0194006 A1
(43) Pub. Date: Aug. 14, 2008

(54) METHODS OF RELEASING SPOROCYSTS FROM OOCYSTS USING CONTROLLED SHEAR FORCES

(75) Inventors: James Earl Hutchins, Durham, NC (US); Kerrianne Wilson, Chester Springs, PA (US); Angela Hartman, Durham, NC (US); Kelly Michelle Harris, Wake Forest, NC (US)

Correspondence Address: PFIZER INC;Steve T. Zelson, 150 EAST 42ND STREET, 5TH FLOOR - STOP 49, NEW YORK, NY, 10017-5612, US

(73) Assignee: Embrex, Inc.

(21) Appl. No.: 12/020,955

(22) Filed: Jan. 28, 2008

Related U.S. Application Data
(60) Provisional application No. 60/900,233, filed on Feb. 8, 2007.

(51) Int. Cl. C12N 1/10

(52) U.S. Cl. 435/258.4

(57) ABSTRACT

Methods of releasing sporocysts/sporozoites from oocysts are provided wherein a solution of oocysts is subjected to controlled shear forces sufficient to rupture the oocysts walls and release sporocysts/sporozoites therefrom. A solution of oocysts is passed through a Microfluidizer® processor chamber unit under defined conditions of chamber diameter, chamber geometry, and pressure. Oocysts impact the wall of the chamber and are subjected to controlled, high shear forces, tearing open the oocyst wall and releasing the sporocysts/sporozoites intact.

### RELATED APPLICATION

This application claims the benefit of and priority to U.S. Provisional Patent Application No. 60/900,233, filed Feb. 8, 2007, the disclosure of which is incorporated herein by reference as if set forth in its entirety.

### FIELD OF THE INVENTION

The present invention relates generally to oocysts and, more particularly, to methods of releasing sporocysts from oocysts.

### BACKGROUND

Coccidiosis of poultry is a disease caused by protozoan parasites of the genus Eimeria. Oocysts of Eimeria species are ubiquitous in the environment and persist for many months in poultry litter. Ingestion of oocysts leads to infection of the various regions of the intestinal tract in a species-specific manner. The organism proliferates in the intestine over a period of several days, resulting in the excretion of the next generation of oocysts in the feces. Multiple cycles of infection lead to immunity, and when the infection is presented to a flock early and in a uniform dosage among the flock, the immunity developed over several cycles of exposure can be quite robust.

In contrast, when birds are not presented with the infection in a uniform manner, situations may arise in which naive birds are subject to sudden, massive infection, leading to poor performance in terms of feed conversion and weight gain, and a high risk of secondary infections. Currently, the most common method used for control of coccidiosis in the poultry industry is not vaccination, but rather the administration of anticoccidial drugs in the feed. The low rate of vaccine use is often attributed to uncertainty in the uniformity in dosing via the feed or water at the growout facility or by spray cabinet vaccination at the hatchery, which are the traditional routes and times of administration. There is increasing interest in improving the uniformity of vaccine delivery during administration at the hatchery and thereby providing more uniform protection within the flock.

Recently, in ovo vaccination techniques have been found applicable to administration of a live oocyst-based coccidiosis vaccine (see, e.g., U.S. Pat. No. 6,500,438; U.S. Pat. No. 6,495,146; and U.S. Pat. No. 6,627,205; all to Pfizer, Inc.). The in ovo route of administration provides a convenient method of delivering a uniform dose of vaccine to each embryo while it is still in the egg. Delivery of avian vaccines in ovo is currently practiced for approximately 85% of the 9 billion broiler birds produced in the United States each year and in a growing percentage of the 21 billion broiler birds produced outside of the United States each year (see, e.g., U.S. Pat. No. 4,458,630 to the United States government). Therefore, the potential market for a live, in ovo-delivered coccidiosis vaccine is considerably larger than the current market for post hatch-delivered coccidiosis vaccines.

Eimeria oocysts contain four sporocysts within the protective oocyst wall. Sporocysts may be used for various purposes, including vaccines, viability testing, sporozoite production, etc. Conventional methods of cracking oocysts and releasing sporocysts utilize glass beads. Shaking oocysts with glass beads causes the oocyst wall to crack and release the sporocysts held therewithin. However, considerable shaking is generally required to crack a high percentage of the oocysts, and the continued shaking action may degrade previously released sporocysts. Similar problems exist with other conventional methods of releasing sporocysts, such as using a tissue grinder to release sporocysts. Sporocysts released early in the process can be destroyed by continuing the grinding process.

Other conventional methods of releasing sporocysts from oocysts involve chemically releasing sporocysts from oocysts. Typically, oocysts are suspended in a buffer containing, for example, CO₂ gas. Cysteine hydrochloride may also be included. This process reduces disulfide bonds in the micropyle region of the oocyst. Eventually, the sporocysts may be released through the loosened micropyle cap. Unfortunately, chemical release alone is not always an efficient method for sporocyst release.

As such, conventional methods of releasing sporocysts from sporulated oocysts are inefficient and yield only a fraction of the potential viable sporocysts available. Accordingly, a need exists for improved ways of releasing sporocysts from oocysts and that overcome the problems associated with the conventional methods.

### SUMMARY

In view of the above discussion, methods of releasing sporocysts from oocysts are provided wherein a solution of oocysts is subjected to controlled shear forces sufficient to rupture the oocyst walls and release viable sporocysts therefrom. According to some embodiments of the present invention, an aqueous solution of oocysts is passed through one or more Microfluidizer® processor chamber units under defined conditions of chamber diameter, chamber geometry, and pressure. Oocysts impact the wall of the chamber and are subjected to controlled, high shear forces, tearing open the oocyst wall and releasing the sporocysts intact. This method is particularly effective in releasing sporocysts because a high percentage of oocyst walls can be cracked allowing a high percentage of sporocysts to be recovered. Moreover, little damage is done to released sporocysts allowing a high percentage of the recovered sporocysts to remain viable.

In some embodiments, the oocysts are treated to weaken the walls thereof prior to subjecting the solution to controlled shear forces. For example, the oocysts may be thermally treated, chemically treated, enzymatically treated, or may be subjected to various combinations of thermal, chemical and enzymatic treatment.

In some embodiments, the recovered sporocysts are cryopreserved for storage. In some embodiments, the recovered sporocysts are used to prepare vaccine and/or a diagnostic assay.

In some embodiments, sporozoites are excysted from the recovered sporocysts. These sporozoites may be used to prepare a vaccine and/or a diagnostic assay.

Exemplary oocysts from which sporocysts may be recovered, according to embodiments of the present invention, are Eimeria oocysts, such as Eimeria oocysts are selected from the group consisting of E. maxima oocysts, E. mitis oocysts, E. tenella oocysts, E. acervulina oocysts, E. brunetti oocysts, E. necatrix oocysts, E. praecox oocysts, E. mivati oocysts, and any combination thereof; Eimeria oocysts selected from the group consisting of E. meleagrimitis oocysts, E. adenoeides oocysts, E. gallopavonis oocysts, E. dispersa oocysts, E. innocua oocysts, and E. subrotunda oocysts, and any combination thereof; Eimeria oocysts selected from the group consisting of E. zuernii oocysts, E. bovis oocysts, and any combination thereof; Eimeria oocysts selected from the group consisting of E. ahsata oocysts, E. bakuensis oocysts, E. crandallis oocysts, E. faurei oocysts, E. granulosa oocysts, E. intricata oocysts, E. marsica oocysts, E. ovinoidalis oocysts, E. pallida oocysts, E. parva oocysts, E. weybridgensis oocysts, and any combination thereof; and Eimeria oocysts selected from the group consisting of E. intestinalis oocysts, E. vejdovskyi oocysts, E. piriformis oocysts, E. coecicola oocysts, E. irresidua oocysts, E. flavescens oocysts, E. exigua oocysts, E. magna oocysts, E. perforans oocysts, E. media oocysts, E. stiedai oocysts, and any combination thereof.

In some embodiments, methods of releasing sporozoites from oocysts are provided wherein a solution of oocysts is subjected to controlled shear forces sufficient to rupture the oocyst walls and release sporozoites therefrom. According to some embodiments of the present invention, an aqueous solution of oocysts is passed through one or more Microfluidizer® processor chamber units under defined conditions of chamber diameter, chamber geometry, and pressure. Oocysts impact the wall of the chamber and are subjected to controlled, high shear forces, tearing open the oocyst wall and releasing the sporozoites intact.

Embodiments of the present invention are advantageous over conventional methods. For example, embodiments of the present invention produce repeatable, consistent yields of sporocysts. In contrast, conventional glass bead and tissue grinder methods may be subject to variations in the way they are performed by an operator and may lead to non-consistent yields of sporocysts. In addition, embodiments of the present invention are scalable allowing economical production of sporocysts on a large scale. In contrast, conventional glass bead and tissue grinding methods are not readily adapted to large-scale production.

### BRIEF DESCRIPTION OF THE DRAWINGS

FIGS. 1-2 are flow charts of operations for releasing sporocysts from oocysts, according to some embodiments of the present invention.

FIG. 3 is a block diagram of a Microfluidizer® processor, according to some embodiments of the present invention.

FIG. 4 is a flow chart of operations for processing released sporocysts, according to some embodiments of the present invention.

FIG. 5 is a flow chart of operations for processing released sporocysts and excysting sporozoites from the released sporocysts, according to some embodiments of the present invention.

### DETAILED DESCRIPTION

The present invention now is described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. All publications, patent applications, patents, and other references mentioned herein are incorporated herein by reference in their entireties.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity. The sequence of operations (or steps) is not limited to the order presented in the claims or figures unless specifically indicated otherwise.

Embodiments of the present invention are suitable for releasing sporocysts from oocysts for medical and veterinary uses, as well as for diagnostic and/or research purposes. The oocysts can be from a protozoan that infects any animal subject, including mammalian and avian subjects. The terms “animal” and “animal subjects” include but are not limited to mammalian and/or avian subjects. Suitable mammalian subjects include but are not limited to primate subjects (e.g., human subjects and non-human primate subjects such as simian), porcine, bovine (e.g., cattle), caprine, equine, feline, ovine, canine, murine (e.g., mouse, rat) and lagomorph subjects.

The terms “avian” and “avian subjects” (i.e., “bird” and “bird subjects”), as used herein, are intended to include males and females of any avian species, but are primarily intended to encompass poultry that are commercially raised for eggs, meat or as pets. Accordingly, the terms “avian” and “avian subject” are particularly intended to encompass but not be limited to chickens, turkeys, ducks, geese, quail, pheasant, parakeets, parrots, cockatoo, cockatiel, ostrich, emu and the like. In particular embodiments, the avian subject is a chicken or a turkey subject. As used herein, an “avian” or “avian subject” can refer to an avian embryo in ovo or an avian post hatch.

The terms “cryopreserve” and “cryopreserving” are well understood by those of skill in the art of the present invention and refer to preserving cells and other material by freezing and storing at very low temperatures.

The present invention relates generally to methods of releasing sporocysts from protozoan oocysts. Such methods find use, for example, in methods of manufacturing vaccines. Many protozoa form a life stage designated as an “oocyst.” The invention can be practiced to release sporocysts from oocysts of any species of protozoa containing sporocysts, including but not limited to Eimeria, Cyclospora, Toxoplasma, Neospora and Isospora.

The present invention may also relate to methods of releasing sporozoites from protozoan oocysts. Some protozoa form a life stage designated as an “oocyst” but may contain sporozoites within the oocyst and do not produce sporocysts. Such methods find use, for example, in methods of releasing sporozoites from oocysts including but not limited to cell-line infection, infectivity assays, manufacturing vaccines, or diagnostic assays. The invention may be practiced to release sporozoites from oocysts of any species of parasite that contains sporozoites within the oocyst, including but not limited to Cryptosporidium and Plasmodium.

The terms “protozoa,” “oocyst,” “sporocyst,” “sporozoite” and “merozoite” have their accepted meanings in the art. Unless indicated otherwise, these terms are intended to refer to live (i.e., viable) protozoa, oocysts, sporocysts, sporozoites and merozoites, including attenuated forms, although those skilled in the art will appreciate that vaccines can be formulated using killed protozoa, oocysts, sporocysts, sporozoites and merozoites. It is understood by those skilled in the art that killed vaccines are usually prepared by first purifying the live organism. Also encompassed herein are genetically modified protozoa, oocysts, sporocysts, sporozoites and merozoites.

The term “Eimeria” indicates one or more species of the genus Eimeria. The term “Eimeria” includes but is not limited to strains or species of Eimeria that infect birds (e.g., chicken, turkey) or mammalian (e.g., cattle, sheep or rabbit) species. Such Eimeria species include those that are found in chickens, including, but not limited to, E. tenella, E. acervulina, E. maxima, E. necatrix, E. mitis, E. praecox, E. mivati and E. brunetti; and also those that are found in turkeys, including, but not limited to, E. meleagrimitis, E. adenoeides, E. gallopavonis, E. dispersa, E. innocua, and E. subrotunda, and those that infect cattle such as, but not limited to, E. bovis and E. zuernil; Eimeria species that infect sheep such as, but not limited to, E. ahsata, E. bakuensis, E. crandalis, E. faurei, E. granulosa, E. intricata, E. marsica, E. ovinoidalis, E. pallida, E. parva, E. weybridgensis; and Eimeria species that infect rabbits including, but not limited to, E. intestinalis, E. vejdovskyi, E. piriformis, E. coecicola, E. irresidua, E. flavescens, E. exigua, E. magna, E. perforans, E. media, and E. stiedai. In addition, the term “Eimeria” includes all strains of the foregoing species of Eimeria including, but not limited to, wildtype strains, precocious or otherwise selected strains, attenuated strains, and oocysts that have been attenuated, e.g., by irradiation, chemical treatment and the like. Further, the term “Eimeria” also includes any newly-discovered strains or species of Eimeria. Finally, the term “Eimeria” encompasses live and killed Eimeria, although live Eimeria are intended unless indicated otherwise.

Compositions comprising Eimeria oocysts find use in methods of immunizing birds against coccidiosis. Methods of vaccinating birds against coccidiosis are known in the art, and include in ovo (e.g., U.S. Pat. No. 6,500,438; U.S. Pat. No. 6,495,146; and U.S. Pat. No. 6,627,205; Pfizer Inc.) and post hatch (e.g., U.S. Pat. No. 3,147,186 to Auburn Research Foundation; U.S. Pat. No. 5,055,292 and U.S. Pat. No. 4,438,097, both to National Research Development Corporation) vaccination methods.

The term “protozoa” includes wildtype strains, precocious or otherwise selected strains, attenuated strains, and oocysts that have been attenuated, e.g., by irradiation, chemical treatment and the like. Further, the term “protozoa” also includes any newly-discovered strains or species of protozoans. Finally, the term “protozoa” covers both live and killed protozoa, although live protozoa are intended unless indicated otherwise. The terms “produce,” “producing” or “production” of oocysts, and the like generally refer to the process of harvesting oocysts from an animal and purifying the oocysts from the fecal material.

Methods of producing oocysts, such as Eimeria oocysts, are known in the art (see, e.g., U.S. Pat. No. 3,147,186 to Auburn Research Foundation; U.S. Pat. No. 4,544,548 to Internationale Octrooi Maatschappij “Octropa” B. V.; U.S. Pat. No. 4,863,731 to Unilever Patent Holdings; international patent publications WO 00/50072 to Pfizer, Inc.; WO 03/020917 to Embrex, Inc.; and WO 02/37961 to Novus International, Inc.; Hammond et al., (1944) Amer. J. Vet. Res. 5:70; Hill et al., (1961) J. Parasit. 47:357; Jackson, (1964) Parasitology54:87; Lotze et al., (1961) J. Parasit. 47:588; Schmatz et al., (1984) J. Protozool. 31:181; Whitlock, (1959) Aust. Vet. J. 35:310); Kowalik et al., (1999) Parasitol. Res. 85:496-499.

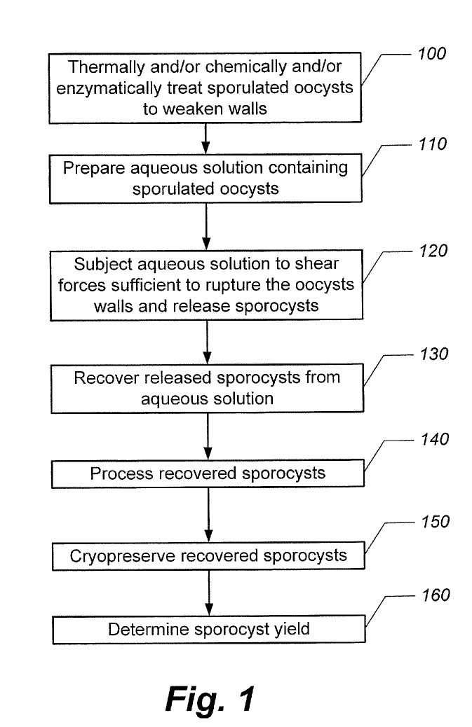

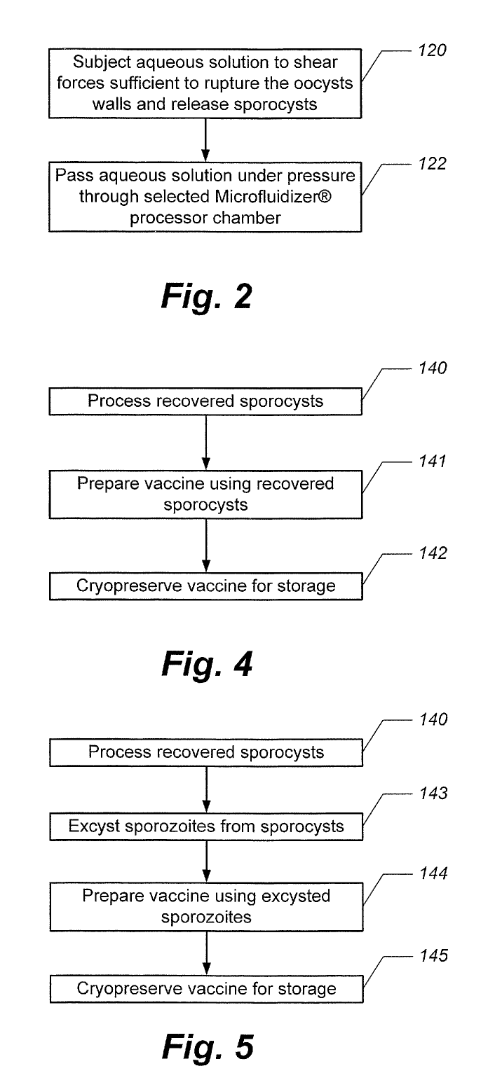

Referring to FIGS. 1-2, methods of releasing sporocysts from sporulated oocysts, according to some embodiments of the present invention, will now be discussed. Initially, sporulated oocysts may be thermally and/or chemically and/or enzymatically pretreated to weaken the walls of the oocysts (Block 100). The weakening caused by thermal, chemical and/or enzymatic treatment causes the oocyst walls to become more susceptible to disruption by shear forces subsequently applied thereto. Exemplary thermal treatments include, but are not limited to, heating the oocysts to between 37° C. and 41° C. for 0.5 hour to 2 hours. Exemplary chemical treatments include, but are not limited to, hypochlorite solution or aqueous solutions containing dissolved carbon dioxide and cysteine hydrochloride, as well as taurodeoxycholic acid. Exemplary enzymatic treatments include, but are not limited to, pepsin and various phospholipases, for example. Thermally, chemically and/or enzymatically pretreating sporulated oocysts is optional and is not required in embodiments of the present invention. Moreover, particular types of sporulated oocysts may not require pretreatment to weaken the walls thereof. Various combinations of thermal, chemical, and/or enzymatic treatment may be used.

An aqueous solution containing sporulated oocysts suspended therein is prepared (Block 110). Exemplary aqueous solutions include, but are not limited to, Hank's balanced salt solution (HBSS), phosphate buffered saline (PBS), RPMI medium, DMEM medium, or the above solutions in combination with a protein such as casein or a protein hydrolysate such as casein hydrolysate or soy protein hydrolysate. The aqueous solution is then subjected to shear forces sufficient to rupture the oocysts' walls and release the sporocysts therefrom (Block 120). The released sporocysts are recovered from the aqueous solution (Block 130). The recovered sporocysts may proceed to subsequent processing (Block 140) and/or may be cryopreserved (Block 150).

Referring to FIG. 2, according to some embodiments of the present invention, subjecting an aqueous solution of oocysts to shear forces sufficient to rupture the oocyst walls and release sporocysts therefrom (Block 120) is performed by passing the aqueous solution under pressure (e.g., between about 2,000 psi and about 6,000 psi) through a Microfluidizer® processor chamber (Block 122). Microfluidizer® processors are available from the Microfluidics Corporation, 30 Ossipee Road, Newton, Mass. Microfluidizer® processors allow high pressure streams of solutions to collide at ultra-high velocities in precisely defined microchannels or chambers. (Other cell disruption equipment can be used including, but not limited to, the Constant Cell Disruption System produced by Constant Systems Ltd., Daventry, Northants, NN11 4SD, England, UK). Embodiments of the present invention are not limited to the use of Microfluidizer® processor chambers.

A Microfluidizer® processor chamber subjects a solution flowing therethrough to combined forces of shear and impact. Each chamber is designed with a fixed-geometry, and is configured to accelerate a product stream to high velocities. The fixed-geometry configuration allows applied shear forces to be precisely controlled and monitored.

Control of shear forces is achieved through the use of defined combinations of chamber geometry, chamber diameter, and applied pressure. Other aspects of the controlled process can include the characteristics and formulation of the solution used, including parameters such as viscosity, specific gravity, chemical composition, osmolality, pH and temperature. Optimized conditions can be determined by those of skill in the art using routine procedures. Consistency of the process may be ensured by performing test runs under defined conditions using buffer alone and determining the flow rate. Tracking the flow rate by such a test over time yields an indication of wear in the equipment or other fault in the system, and corrective action may be taken to return the system to standard operating conditions.

Exemplary Microfluidizer® processor chambers that may be used according to some embodiments of the present invention include chambers with “Y-shaped” and “Z-shaped” configurations. Y-shaped chambers include two entering flow paths that converge at a point and exit as a single flow path. Z-shaped chambers have a single flow path with a Z-shaped path. Chamber configurations for optimal recovery of sporocysts may vary by species. Accordingly, different chamber configurations may be selected for different types of oocysts. In addition, Microfluidizer® processor chambers may be arranged in series.

In addition, Microfluidizer® processor chambers may have different diameters. For example, diameters of between about 75 microns (μm) and about 500 μm may be utilized. Microfluidizer® processor chambers of different diameters may be arranged in series, also.

Applicants have found that chambers having a diameter that is substantially equal to or larger than the diameter of the oocysts in solution are particularly effective. For example, for E. maxima oocysts, which are typically about 20 μm to 40 μm in diameter, a reaction chamber with a diameter of about 300 μm is preferred. However, diameters ranging from about 75 μm to about 400 μm may also be used. Typically, the flow path used is the Z configuration, although a Y configuration or other configuration may also be used. Flow rates normally range from about 500 mL per min to about 2000 mL per minute at pressures ranging from 1000 to 5000 psi. The amount of shear force typically required to release sporocysts from E. maxima oocysts ranges from about 3.00×10⁵ sec⁻¹ per minute to about 1.50×10⁶ sec⁻¹ per minute.

For E. tenella oocysts, which are typically about 15 μm to 25 μm in diameter, and E. acervulina, which are typically about 10 μm to 15 μm in diameter, a Microfluidizer® processor reaction chamber with a diameter of 100 μm is preferred. However, diameters ranging from about 75 μm to about 400 μm may also be used. Typically, the flow path used is the Z configuration, although a Y configuration or other configuration may also be used. Flow rates normally range from about 100 mL per min to about 250 mL per minute at pressures ranging from 1000 to 4000 psi. The amount of shear force typically required to release E. tenella or E. acervulina sporocysts from oocysts ranges from about 1.00×10⁶ sec⁻¹ per minute to about 3.00×10⁶ sec⁻¹ per minute.

For any species of Eimeria oocysts, conditions resulting in lower shear forces may be used to release sporocysts from oocysts, especially when oocysts have been thermally, chemically, or enzymatically pre-treated. Although multiple passes through the chamber may be used, a single pass is preferred.

Embodiments of the present invention are not limited to a particular chamber diameter for a particular oocyst. Embodiments of the present invention may utilize chambers having all types of configurations and diameters.

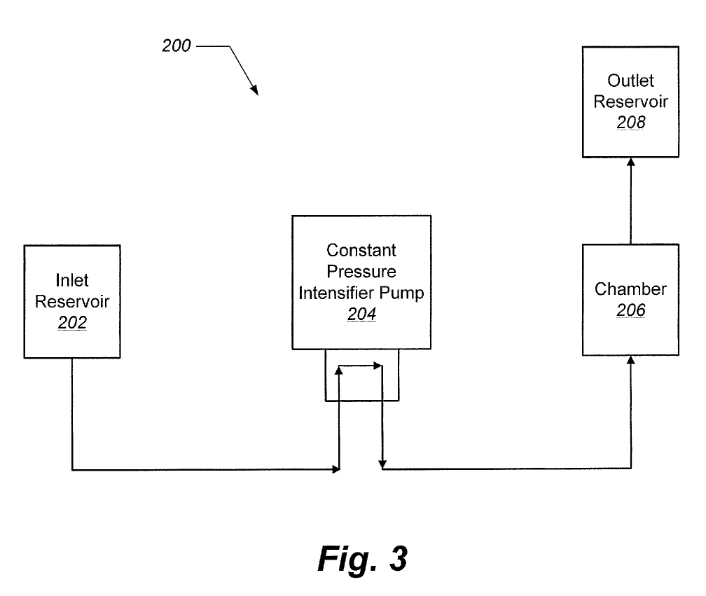

Referring to FIG. 3, a Microfluidizer® processor 200 is illustrated. The illustrated Microfluidizer® processor 200 includes an inlet reservoir 202 containing an aqueous solution of sporulated oocysts. The aqueous solution is pressurized and pumped via a pump 204 (e.g., constant pressure intensifier pump, etc.) through a chamber 206 (e.g., a Y-shaped, Z-shaped chamber, etc.). The oocysts in the pressurized aqueous solution are subjected to shear and impact forces in the chamber 206 causing sporocysts to be released from the oocyst walls. The aqueous solution containing the released sporocysts is collected in the outlet reservoir 208.

Referring back to FIG. 1, according to some embodiments of the present invention, a percentage of sporocysts released and recovered from oocysts (i.e., sporocyst yield) may be determined (Block 160). Sporocyst yield may be assessed, for example, via microscopy. Sporocysts deemed recovered by microscopy may be either viable or non-viable or a mixture thereof. However, the viability status may not be determinable by microscopy alone. Accordingly, a determination of a percentage of released sporocysts that are viable (i.e., viability yield) may be performed, also. Determining viability yield may require an in vivo procedure. Appropriate in vivo procedures may include administering sporocyst preparations to avian subjects via oral gavage and comparing the resulting oocyst output with that obtained by administering intact oocysts of equivalent number.

Referring to FIG. 4, recovered sporocysts may be processed in various ways and for various purposes. For example, vaccines may be prepared from recovered sporocysts (Block 141) and cryopreserved for storage (Block 142). Referring to FIG. 5, according to some embodiments of the present invention, recovered sporocysts may be processed to release (i.e., excyst) sporozoites therefrom (Block 143). The released sporozoites may be used to prepare vaccines (Block 144) or may be used for some other purpose. Vaccines prepared from excysted sporozoites may be cryopreserved for storage (Block 145).

Any type of oocyst may be processed in accordance with embodiments of the present invention. Particularly suitable oocysts are Eimeria oocysts including, but not limited to, E. maxima oocysts, E. mitis oocysts, E. tenelfa oocysts, E. acervulina oocysts, E. brunetti oocysts, E. necatrix oocysts, E. praecox oocysts, E. mivati oocysts, and any combination thereof, E. meleagrimitis oocysts, E. adenoeides oocysts, E. gallopavonis oocysts, E. dispersa oocysts, E. innocua oocysts, and E. subrotunda oocysts, and any combination thereof, E. zuernii oocysts, E. bovis oocysts, and any combination thereof, E. ahsata oocysts, E. bakuensis oocysts, E. crandallis oocysts, E. faurei oocysts, E. granulosa oocysts, E. intricata oocysts, E. marsica oocysts, E. ovinoidalis oocysts, E. paflida oocysts, E. parva oocysts, E. weybridgensis oocysts, and any combination thereof, E. intestinalis oocysts, E. vejdovskyi oocysts, E. piriformis oocysts, E. coecicola oocysts, E. irresidua oocysts, E. flavescens oocysts, E. exigua oocysts, E. magna oocysts, E. perforans oocysts, E. media oocysts, E. stiedai oocysts, and any combination thereof.

Having described the present invention, the same will be explained in greater detail in the following examples, which are included herein for illustration purposes only, and which are not intended to be limiting to the invention.

### EXAMPLE 1

A series of experiments were performed to map out sporocyst recovery and oocyst cracking over a broad range of parameter values. Each experiment used a total of approximately 2×10⁸ sporulated oocysts in 500 mL HBSS (4×10⁵ sporulated oocysts per mL); a sub-sample was taken for enumeration prior to Microfluidizer® processor treatment. Chamber diameters to be tested varied according to species: 1) E. maxima: 200, 300, 400 micron diameter chambers; 2) E. tenella: 200, 300 micron diameter chambers; 3) E. acervulina: 125 micron diameter chamber. Single pass mode was used. Pressures ranging from 2,000 psig to 6,000 psig were used, depending on the study. After passing through the Microfluidizer® processor, the total volume was adjusted to 1 L with HBSS, the sample was mixed, and a subsample was taken for enumeration. Typically, three replicate runs were made using each set of conditions.

Percent sporocysts recovered and percent oocysts cracked were determined for each experiment using hemacytometer counts. Ideally, both percent sporocysts recovered and percent oocysts cracked would be 100%. E. acervulina Sporocyst Release using Microfluidizer® processor

| | Pressure | | | |
|---|---|---|---|---|
| Chamber | 5000 psig | | 6000 psig | |
| diameter | % Sporocyst | % Oocysts | % Sporocyst | % Oocysts |
| (μm) | Recovery | Cracked | Recovery | Cracked |
| 125 Y | 54.26 | 48.18 | 49.46 | 75.61 |

Conclusions for E. acervulina 1 Microfluidizer® experiments:

1) E. acervulina percent oocysts cracked were observed to increase with increasing pressure using the 125 micron chamber.

2) The best results for sporocyst recovery were obtained using the 125 micron chamber at 5000 psig (54% recovery).

E. maxima 1 sporocyst release using Microfluidizer® processor

| Cham- | Pressure | | | | | |
|---|---|---|---|---|---|---|
| ber | 2000 psig | | 3000 psig | | 4000 psig | |
| Diam- | % | % | % | % | % | % |
| eter | Sporocyst | Oocysts | Sporocyst | Oocysts | Sporocyst | Oocysts |
| (μm) | Recovery | Cracked | Recovery | Cracked | Recovery | Cracked |
| 200 Z | 96.83 | 47.55 | 131.06 | 64.34 | 119.50 | 83.45 |
| 300 Z | 109.55 | 79.07 | 106.03 | 89.91 | 95.33 | 93.98 |
| 400 Z | 96.69 | 55.73 | 116.93 | 80.90 | 129.85 | 87.74 |

Values are averages of at least three replicate single-pass runs.

Conclusions for E. maxima 1 Microfluidizer® experiments:

1) The sporocyst release process worked reasonably well for nearly all combinations of chamber diameter and pressure tested.

2) The combination of the 300 micron chamber and 3000 psig pressure provided suitable initial standard conditions for production of E. maxima 1 sporocysts using a Microfluidizer® processor.

E. maxima 2 Sporocyst Release using Microfluidizer® processor

| Cham- | Pressure | | | | | |
|---|---|---|---|---|---|---|
| ber | 2000 psig | | 3000 psig | | 4000 psig | |
| Diam- | % | % | % | % | % | % |
| eter | Sporocyst | Oocysts | Sporocyst | Oocysts | Sporocyst | Oocysts |
| (μm) | Recovery | Cracked | Recovery | Cracked | Recovery | Cracked |
| 200 Z | 114.18 | 93.99 | 104.58 | 87.07 | 73.71 | 95.23 |
| 300 Z | 93.47 | 85.95 | 115.43 | 91.01 | 110.31 | 92.80 |
| 400 Z | 98.12 | 86.63 | 110.04 | 94.24 | 111.32 | 97.84 |

Values are averages of at least three replicate single-pass runs.

Conclusions for E. maxima 2 Microfluidizer® processor experiments:

1) The sporocyst release process worked reasonably well for nearly all combinations of chamber diameter and pressure tested.

2) The combination of the 300 micron chamber and 3000 psig pressure was considered optimal.

E. tenella 1 Sporocyst Release using Microfluidizer® processor

| Cham- | Pressure | | | | | |
|---|---|---|---|---|---|---|
| ber | 2000 psig | | 3000 psig | | 4000 psig | |
| Diam- | % | % | % | % | % | % |
| eter | Sporocyst | Oocysts | Sporocyst | Oocysts | Sporocyst | Oocysts |
| (μm) | Recovery | Cracked | Recovery | Cracked | Recovery | Cracked |
| 125 Y | 50.33 | 54.16 | 68.46 | 71.88 | 98.18 | 84.04 |
| 200 Z | 19.86 | 13.95 | 43.80 | 39.74 | 56.30 | 62.03 |

Values are averages of at least three replicate single-pass runs.

Conclusions for E. tenella 1 Microfluidizer® processor experiments:

1) E. tenella sporocyst recovery and percent oocysts cracked were observed to increase with increasing pressure for both chambers tested, as expected.

2) The best results were obtained using the 125 micron chamber at 4000 psig.

Embodiments of the present invention provide a repeatable, scalable alternative for recovery of sporocysts over conventional tissue grinder, glass bead, and chemical release methods. Essentially quantitative recovery of sporocysts from E. maxima 1 and E. maxima 2 is provided. Nearly quantitative recovery of E. tenella sporocysts is observed. Recovery of E. acervulina oocysts is about 40-50%.

### EXAMPLE 2

Using cell disruption equipment to release sporocysts requires optimization of release conditions to ensure viability of released sporocysts. Viability may be assessed by providing a dose of sporocysts to birds and by enumerating the associated oocyst output resulting from the infection. Under varying conditions of cell disruption equipment chamber geometry and pressure, it is possible to efficiently release sporocysts from oocysts in a non-viable condition. That is, the infection produced in birds upon administration of the sporocysts may be lessened as compared to that achieved using sporocysts released by traditional methods such as glass beads. Conditions of chamber geometry and pressure must be carefully assessed to ensure that viable sporocysts are produced.

Sporocysts were released from E. acervulina oocysts using either glass beads or the Microfluidizer® processor configured with a 100Z chamber at 2000 psi or 5000 psi. Sporocysts released by each method were then administered to chickens in a dose response model using 1000, 3000, and 5000 sporocysts per dose. Three replicate pens were used for each treatment, with nine birds per replicate. An oocyst control at 1250 sporulated oocysts per dose representing 5000 sporocysts was also included in the test. Oocysts were collected from days 4 to 7 post gavage into a solution of 10% citric acid, 0.75% hydrogen peroxide, and 0.25% propionic acid in water. Feces containing oocysts were brought to a uniform volume using water, blended, and subsampled. Oocysts were enumerated using the McMaster's method. Results are shown in the table below:

Effect of Pressure on the Viability of E. acervulina Sporocysts

| | | Mean Oocyst Output |
|---|---|---|
| Dose | Treatment | per Bird |
| 1250 | Sporulated oocyst control | 5.18 × 10⁷ |
| 1000 | Microfluidizer ® at 2000 psi | 1.22 × 10⁷cd |
| | Microfluidizer ® 5000 psi | 2.85 × 10⁶ e |
| | Glass Beads | 1.58 × 10⁷c |
| 3000 | Microfluidizer ® at 2000 psi | 3.93 × 10⁷b |
| | Microfluidizer ® 5000 psi | 6.07 × 10⁶de |
| | Glass Beads | 5.30 × 10⁷ab |
| 5000 | Microfluidizer ® at 2000 psi | 1.00 × 10⁸ab |
| | Microfluidizer ® 5000 psi | 7.61 × 10⁶cd |
| | Glass Beads | 1.11 × 10⁸a |

Statistical comparisons were made between treatments receiving sporocysts.
Different letters in common represent significant differences to p = .05.

Results indicate that sporocysts produced using the Microfluidizer® processor at 2000 psi were as viable as sporocysts produced using the traditional glass bead method at each dose, while sporocysts produced at 5000 psi using the Microfluidizer® processor were significantly less viable than those produced using the glass bead method at each dose. The range of pressure which yields viable sporocysts must therefore be determined experimentally.

### EXAMPLE 3

The smaller Eimeria species, including E. acervulina (˜12 micron length) and E. tenella (˜20 micron length) are observed to be less susceptible to shear than the larger E. maxima species (˜40 microns length). Generating higher levels of shear by increasing the pressure in the Microfluidizer® system can improve microscopic yield of sporocysts from oocysts for any species, but is especially required for efficient release of the smaller species; however, increasing pressure can also decrease viability. Lowering pressure to improve viability inherently sacrifices yield. Pre-treatments have been developed to condition the wall of the oocysts to provide both improved microscopic yield and improved viability.

A study was performed to examine the effect of pre-treatment of oocysts using a combination of a bile salt (taurodeoxycholic acid (TDCA), an anaerobic environment (bubbled carbon dioxide), and a warm temperature (37° C. for 1 h). The objectives of the study were to determine the in vitro recovery (via microscopy) and the in vivo viability (via oocyst output) of released sporocysts.

The following treatment groups were established: (1) E. acervulina sporulated oocyst control; 1000 sporulated oocysts per dose; (2) Glass bead-produced sporocyst control; 4000 sporocysts per dose; (3) Microfluidizer®-produced sporocyst control without pretreatment; 100 μm chamber; 2,000 psi; 4000 sporocysts per dose; (4) Microfluidizer®-produced sporocysts with pre-treatment including 0.75% taurodeoxycholic acid (TDCA) and bubbled carbon dioxide at 37° C. for 1 h; 100 μm chamber; 2,000 psi; 4000 sporocysts per dose; (5) Microfluidizer®-produced sporocysts with pre-treatment including 0.75% TDCA and bubbled carbon dioxide at 37° C. for 1 h; 100 μm chamber; 3,000 psi; 4000 sporocysts per dose; (6) Microfluidizer®-produced sporocysts with pre-treatment including 0.75% TDCA and bubbled carbon dioxide at 37° C. for 1 h; 100 μm chamber; 4,000 psi; 4000 sporocysts per dose. Residual intact oocysts and oocyst shells were removed from the sporocyst preparations using Percoll. Sporocyst doses were delivered to birds at 4,000 sporocysts per dose to provide doses equivalent to the 1,000 oocyst per dose control, as each oocyst contains four sporocysts.

Recovery of sporocysts from oocysts was evaluated microscopically. Results for the Microfluidizer®-produced materials are summarized in the table below:

Microscopic Recovery of E. acervulina Sporocysts using Pre-Treatment

| | | Microfluidizer ® | Sporocysts |
|---|---|---|---|
| | | Pressure | Recovered |
| Treatment | Pre-Treatment | (psi) | (%) |
| 3 | No | 2000 | 5.42 |
| 4 | Yes | 2000 | 33.70 |
| 5 | Yes | 3000 | 54.90 |
| 6 | Yes | 4000 | 65.48 |

Pre-treatment improved recovery of sporocysts approximately six-fold at 2000 psi, and at increasing pressures, further improvement in microscopic recovery was observed.

For the in vivo assessment, each treatment used 5 replicate pens with nine birds per pen. Treatments were administered via oral gavage to the crop. Feces were collected into a solution of 10% citric acid, 0.75% hydrogen peroxide, and 0.25% propionic acid in water from days 4 to 7 post gavage. Feces containing oocysts were brought to a uniform volume, blended, and subsampled. Oocysts were enumerated using the McMaster's method.

The viability of sporocysts produced using the stated release methods was assessed by comparing oocyst output per bird relative to the glass bead-produced sporocyst control. Results are summarized in the table below:

Viability of E. acervulina Sporocysts Released from Pre-Treated Oocysts

| | | | | | | Viability |
|---|---|---|---|---|---|---|
| | | | | | | as |
| | | | | | Mean | Percent |
| | | | | Microfluidizer ® | Oocyst | of Glass |
| | Eimeria | Pre- | Release | Pressure | Output | Bead |
| Treatment | Life Stage | Treatment | method | (psi) | per Bird | Control |
| 1 | Sporulated | NA | NA | NA | 3.00 × 10⁷ | NA |
| | oocysts | | | | | |
| 2 | Sporocysts | NA | Glass Beads | NA | 2.97 × 10⁷ | 100.0 |
| 3 | Sporocysts | No | Microfluidizer ® | 2000 | 2.58 × 10⁷ | 86.9 |
| 4 | Sporocysts | Yes | Microfluidizer ® | 2000 | 4.05 × 10⁷ | 136.4 |
| 5 | Sporocysts | Yes | Microfluidizer ® | 3000 | 2.41 × 10⁷ | 81.1 |
| 6 | Sporocysts | Yes | Microfluidizer ® | 4000 | 4.03 × 10⁷ | 136.0 |

There were no significant differences among treatments in mean oocyst output per bird (p>0.05). Pre-treatment provided improved viability of released sporocysts across a wide range of pressure. In some cases, the viability of sporocysts released from pretreated oocysts by the Microfluidizer® method was numerically higher than that of the glass bead-released sporocysts. The overall effect of pre-treatment was thus two-fold, improving both recovery from oocysts during the release step and viability.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

That which is claimed is:

1. A method of releasing sporocysts from oocysts, the method comprising:
preparing a solution containing oocysts suspended therein;
subjecting the solution to controlled shear forces sufficient to rupture walls of the oocysts and release viable sporocysts therefrom; and
recovering the released viable sporocysts from the solution.

2. The method of claim 1, wherein subjecting the solution to controlled shear forces sufficient to rupture the oocysts walls and release sporocysts therefrom comprises passing the solution under pressure through a Microfluidizer® processor chamber one or more times.

3. The method of claim 1, wherein the solution comprises an aqueous solution.

4. The method of claim 3, wherein the aqueous solution comprises a solution selected from the group consisting of: Hank's balanced salt solution (HBSS), phosphate buffered saline (PBS), RPMI medium, and DMEM medium.

5. The method of claim 1, wherein the amount of shear force used to release sporocysts from E. maxima oocysts ranges from about 3.00×10⁵ sec⁻¹ per minute to about 1.50×10⁶ sec⁻¹ per minute.

6. The method of claim 1, wherein the amount of shear force used to release E. tenella or E. acervulina sporocysts from oocysts ranges from about 1.00×10⁶ sec⁻¹ per minute to about 3.00×10⁶ sec⁻¹ per minute.

7. The method of claim 2, wherein the solution passing through the Microfluidizer® processor chamber is pressurized to between about 1,000 psi and about 6,000 psi.

8. The method of claim 7, wherein the Microfluidizer® processor chamber has a diameter of between about 75 microns to about 400 microns, and wherein the solution has a flow rate of between about 100 mL to about 2,000 mL.

9. The method of claim 1, wherein the Microfluidizer® processor chamber has a Z-shaped configuration.

10. The method of claim 1, wherein the Microfluidizer® processor chamber has a Y-shaped configuration.

11. The method of claim 1, wherein the Microfluidizer® processor includes a plurality of chambers, and further comprising selecting a chamber having a diameter that is substantially equal to or larger than a diameter of the oocysts and passing the solution under pressure through the selected chamber.

12. The method of claim 1, wherein the Microfluidizer® processor includes a plurality of chambers in series, each chamber having a respective different diameter, and further comprising selecting a chamber having a diameter that is substantially equal to or larger than a diameter of the oocysts and passing the solution under pressure through the selected chamber.

13. The method of claim 1, comprising thermally treating the oocysts to weaken the walls thereof prior to subjecting the solution to controlled shear forces.

14. The method of claim 1, comprising chemically treating the oocysts to weaken the walls thereof prior to subjecting the solution to controlled shear forces.

15. The method of claim 1, comprising enzymatically treating the oocysts to weaken the walls thereof prior to subjecting the solution to controlled shear forces.

16. The method of claim 1, comprising using a combination of thermal, chemical, or enzymatic treatment of oocysts to weaken the walls thereof prior to subjecting the solution to controlled shear forces.

17. The method of claim 1, further comprising cryopreserving the recovered sporocysts.

18. The method of claim 1, further comprising preparing a vaccine and/or a diagnostic assay using the recovered sporocysts.

19. The method of claim 1, further comprising excysting sporozoites from the recovered sporocysts.

20. The method of claim 19, further comprising preparing a vaccine using the excysted sporozoites.

21. The method of claim 1, further comprising determining a percentage of sporocysts released from the oocysts.

22. The method of claim 1, further comprising determining a percentage of released sporocysts that are viable.

23. The method of claim 1, wherein the oocysts are Eimeria oocysts.

24. The method of claim 23, wherein the Eimeria oocysts are selected from the group consisting of E. maxima oocysts, E. mitis oocysts, E. tenella oocysts, E. acervulina oocysts, E. brunetti oocysts, E. necatrix oocysts, E. praecox oocysts, E. mivati oocysts, and any combination thereof.

25. The method of claim 23, wherein the Eimeria oocysts are selected from the group consisting of E. meleagrimitis oocysts, E. adenoeides oocysts, E. gallopavonis oocysts, E. dispersa oocysts, E. innocua oocysts, and E. subrotunda oocysts, and any combination thereof.

26. The method of claim 23, wherein the Eimeria oocysts are selected from the group consisting of E. zuernii oocysts, E. bovis oocysts, and any combination thereof.

27. The method of claim 23, wherein the Eimeria oocysts are selected from the group consisting of E. ahsata oocysts, E. bakuensis oocysts, E. crandallis oocysts, E. faurei oocysts, E. granulosa oocysts, E. intricata oocysts, E. marsica oocysts, E. ovinoidalis oocysts, E. pallida oocysts, E. parva oocysts, E. weybridgensis oocysts, and any combination thereof.

28. The method of claim 23, wherein the Eimeria oocysts are selected from the group consisting of E. intestinalis oocysts, E. vejdovskyi oocysts, E. piriformis oocysts, E. coecicola oocysts, E. irresidua oocysts, E. flavescens oocysts, E. exigua oocysts, E. magna oocysts, E. perforans oocysts, E. media oocysts, E. stiedai oocysts, and any combination thereof.

29. A method of releasing sporozoites from oocysts, the method comprising:
preparing a solution containing oocysts suspended therein;
subjecting the solution to controlled shear forces sufficient to rupture walls of the oocysts and release viable sporozoites therefrom; and
recovering the released viable sporozoites from the solution.

30. The method of claim 29, wherein subjecting the solution to controlled shear forces sufficient to rupture the oocysts walls and release sporozoites therefrom comprises passing the solution under pressure through a Microfluidizer® processor chamber one or more times.

31. The method of claim 29, wherein the solution comprises an aqueous solution.

32. The method of claim 29, comprising treating the oocysts to weaken the walls thereof prior to subjecting the solution to controlled shear forces.

33. The method of claim 29, further comprising cryopreserving the recovered sporozoites.

34. The method of claim 29, further comprising preparing a vaccine and/or a diagnostic assay using the recovered sporozoites.
